SEQUENCE LISTING (38 sequences)

SEQ ID NO: 1 - DNA, 26 bp, Artificial Sequence
  Restriction sites
ggatccatgcaagcttatgcgatatc

SEQ ID NO: 2 - DNA, 57 bp, Artificial Sequence
  Promoter Ptrc01
gagctgttgacaattaatcatccggctcgtataatgtgtggaataaggaggtatatc

SEQ ID NO: 3 - DNA, 1689 bp, Artificial Sequence
  synthetic gene
atgacctggctggaaccgcagattaaaagccagctgcagagcgaacgtaaagattgggaagcgaacgaagtgggcgcgtttctgaaaaaagcgccggaacgtaaagaacagtttcataccattggcgattttccggtgcagcgtacctataccgcagcggatattgcggataccccgctggaagatattggcctgccgggtcgttatccgtttacccgtggcccgtatccgaccatgtatcgtagccgtacctggaccatgcgtcagattgcgggctttggcaccggcgaagataccaacaaacgctttaaatatctgattgcgcagggccagaccggcattagcaccgattttgatatgccgaccctgatgggctatgatagcgatcatccgatgagcgatggcgaagtgggccgtgaaggcgtggcgattgataccctggccgatatggaagcgctgctggccgatattgatctggaaaaaatcagcgtgagctttaccattaatccgagcgcgtggattctgctggccatgtatgtggcgctgggcgaaaaacgtggctatgatctgaacaaactgagcggcaccgtgcaggcggatattctgaaagaatatatggcgcagaaagaatacatttatccgattgcgccgagcgtgcgcattgtgcgtgatattattacctacagcgcgaaaaacctgaaacgctacaatccgattaacatcagcggctatcatattagcgaagcgggcagcagcccgctgcaggaagcggcgtttacgctggccaacctgattacctatgtgaacgaagtgaccgaaaccggcatgcatgtggatgaatttgcgccgcgtctggcctttttttttgtgagccagggcgatttttttgaagaagtggcgaaatttcgcgcgctgcgtcgttgctatgcgaaaataatgaaagaacgctttggcgcgaaaaatccggaaagcatgcgtctgcgttttcattgccagaccgcagcggcgaccctgaccaaaccgcagtatatggtgaacgtggtgcgtaccagcctgcaggccctgagcgcggttctgggtggtgcgcagagcctgcataccaacggctatgatgaagcgtttgcgattccgaccgaagatgccatgaaaatggcgctgcgtacccagcagattattgcggaagaaagcggcgtggcggatgtgattgatccgctgggcggcagctattatgtggaagcgctgaccaccgaatacgaaaaaaaaatcttcgaaatcctggaagaggtggaaaaacgcggtggcaccattaaactgattgaacagggctggtttcagaaacaaattgcggattttgcgtatgaaaccgcgctgcgtaaacagagcggccagaaaccggtgattggcgtgaaccgttttgtggaaaacgaagaagatgtgaaaatcgaaatccacccgtatgataacaccaccgcggaacgtcagattagccgtacccgtcgtgtgcgtgcggaacgtgatgaagcgaaagtgcaggccatgctggatcagctggttgcggtggcgaaagatgaaagccagaacctgatgccgctgaccattgaactggtgaaagcgggtgcgaccatgggcgatattgtggaaaaactgaaaggcatttggggcacctatcgtgaaaccccggtgttttaa

SEQ ID NO: 4 - DNA, 17 bp, Artificial Sequence
  Intergenic sequence
gaataaggaggtatatt

SEQ ID NO: 5 - DNA, 411 bp, Artificial Sequence
  Synthetic gene
atggatcagattccgattcgtgtgctgctggccaaagtgggcctggatggccatgatcgtggcgtgaaagtggtggcgcgtgcgctgcgtgatgcgggcatggatgtgatttatagcggcctgcatcgtaccccggaagaagtggtgaacaccgcgattcaagaggacgtggatgtgctgggcgtgagcctgctgtctggtgtgcagctgaccgtgtttccgaaaatcttcaaactgctggaagaacgcggtgcgggcgatctgattgtgattgcgggcggtgtgatgccggatgaagatgcggcagcgattcgtaaactgggcgtgcgtgaagtgctgctgcaggataccccaccgcaggcgattattgatagcattcgtgcgctggttgcggcgcgtggtgcgcgttaa

SEQ ID NO: 6 - DNA, 28 bp, Artificial Sequence
  Terminator T7Te
ctggctcaccttcgggtgggcctttctg

SEQ ID NO: 7 - DNA, 27 bp, Artificial Sequence
  Restriction sites
ccccgggatgcggatccatgcgaattc

SEQ ID NO: 8 - DNA, 24 bp, Artificial Sequence
  Oligonucleotide
ccgacagtaagacgggtaagcctg

SEQ ID NO: 9 - DNA, 22 bp, Artificial Sequence
  Oligonucleotide
agcttagtaaagccctcgctag

SEQ ID NO: 10 - DNA, 26 bp, Artificial Sequence
  Restriction sites
ggatccatgcaagcttatgccccggg

SEQ ID NO: 11 - DNA, 100 bp, Artificial Sequence
  Promoter Plac
aagctcactcattaggcaccccaggctttacactttatgcttccggctcgtatgttgtgtggaattgtgagcggataacaatttcacacaggactacaca

SEQ ID NO: 12 - DNA, 1182 bp, Artificial Sequence
  synthetic gene
atgaccgatgtggtgattgtgagcgcagcgcgtaccgcggtgggcaaatttggcggcagcctggccaaaattccggctccggaactgggtgcggtggtgattaaagcggcgctggaacgtgcgggcgtgaaaccggaacaggtgagcgaagtgattatgggccaggtgctgaccgcgggttctggtcagaatccggcacgtcaggcggcgattaaagcgggtctgccggccatggttccggccatgaccattaacaaagtgtgcggcagcggcctgaaggcggttatgctggccgcgaacgcgattatggcgggtgatgcggaaattgtggtggcgggtggccaagaaaatatgagcgcagccccgcatgttctgccgggcagccgtgatggctttcgtatgggtgatgcgaaactggtggataccatgattgtggatggcctgtgggatgtgtataaccagtatcatatgggcattaccgcggaaaacgtggcgaaagaatatggcattacccgtgaagcgcaggatgaatttgcggttggcagccagaacaaagcggaagctgcgcagaaagcgggcaaatttgatgaagaaattgtgccggtgctgattccgcagcgtaaaggcgatccggtggcgtttaaaaccgatgaatttgtgcgtcaaggcgcgaccctggatagcatgtctggtctgaaaccggcgtttgataaagcgggcaccgtgaccgcggcgaatgcgtctggcctgaacgatggtgcagcagcggtggttgtgatgagcgctgcgaaagcgaaagaactgggcctgacgccgctggccaccattaaaagctatgcgaacgcgggcgttgatccgaaagtgatgggcatgggtccggtgccggcgagcaaacgtgccctgagccgtgcggaatggaccccgcaggatctggatctgatggaaatcaacgaagcgtttgcggcgcaggcgctggcagttcatcagcagatgggctgggataccagcaaagtgaacgtgaacggcggtgcgattgcgattggccatccgattggcgcgtctggctgccgtattctggtgaccctgctgcatgaaatgaaacgtcgtgatgcgaaaaaaggcctggccagcctgtgcattggcggcggtatgggtgttgcgctggccgtggaacgtaaataa

SEQ ID NO: 13 - DNA, 74 bp, Artificial Sequence
  Intergenic sequence
ggaaggggttttccggggccgcgcgcggttggcgcggacccggcgacgataacgaagccaatcaaggagtggac

SEQ ID NO: 14 - DNA, 741 bp, Artificial Sequence
  Synthetic gene
atgacccagcgtattgcgtatgtgaccggcggcatgggcggtattggcaccgcgatttgccagcgtctggctaaagatggctttcgcgtggtggccggttgcggtccgaatagcccgcgtcgtgaaaaatggctggaacagcagaaagcgctgggctttgattttattgcgagcgaaggtaacgtggcggattgggatagcaccaaaaccgcgttcgataaagtgaaaagcgaagtgggtgaagtggatgtgctgattaacaacgccggcatcacccgtgatgtggtgtttcgcaaaatgacccgtgccgattgggatgcggtgattgataccaacctgacctccctgtttaacgtgaccaaacaggtgattgatggcatggcggatcgtggctggggccgtattgtgaacattagcagcgtgaacggccagaaaggccagtttggccagaccaactatagcaccgcgaaagcgggcctgcatggctttaccatggcgctggcccaggaagtggcgaccaaaggcgtgaccgtgaacaccgtgtctccgggctatattgcgaccgatatggtgaaagcgattcgtcaggatgtgctggataaaattgtggcgaccattccggtgaaacgtctgggcctgccggaagaaattgcgagcatttgcgcgtggctgtctagcgaagaaagcggctttagcaccggtgcggattttagcctgaacggcggcctgcatatgggctaa

SEQ ID NO: 15 - DNA, 55 bp, Artificial Sequence
  Terminator rrnB
catcaaataaaacgaaaggctcagtcgaaagactgggcctttcgttttatctgtt

SEQ ID NO: 16 - DNA, 26 bp, Artificial Sequence
  Restriction sites
gtatactgcaggatccatgcgaattc

SEQ ID NO: 17 - DNA, 100 bp, Artificial Sequence
  Oligonucleotide
ccaacgcagaccgctgcctggcaggcactacagaaacacttcgatgaaatgaaagacgttacgatcgccgatctttttgctgtaggctggagctgcttcg

SEQ ID NO: 18 - DNA, 100 bp, Artificial Sequence
  Oligonucleotide
gcgccacgctttatagcggttaatcagaccattggtcgagctatcgtggctgctgatttctttatcatctttcagctctgcatatgaatatcctccttag

SEQ ID NO: 19 - DNA, 30 bp, Artificial Sequence
  Oligonucleotide
gcggggcggttgtcaacgatggggtcatgc

SEQ ID NO: 20 - DNA, 30 bp, Artificial Sequence
  Oligonucleotide
cggtatgatttccgttaaattacagacaag

SEQ ID NO: 21 - DNA, 100 bp, Artificial Sequence
  Oligonucleotide
cccagaatctcttttgtttcccgatggaacaaaattttcagcgtgcccacgttcatgccgacgatttgtgcgcgtgccagtgtaggctggagctgcttcg

SEQ ID NO: 22 - DNA, 100 bp, Artificial Sequence
  Oligonucleotide
ggtgcgcgcgtcgcagttatcgagcgttatcaaaatgttggcggcggttgcacccactggggcaccatcccgtcgaaagccatatgaatatcctccttag

SEQ ID NO: 23 - DNA, 20 bp, Artificial Sequence
  Oligonucleotide
gatgctggaagatggtcact

SEQ ID NO: 24 - DNA, 18 bp, Artificial Sequence
  Oligonucleotide
gtgaatgaacggtaacgc

SEQ ID NO: 25 - DNA, 37 bp, Artificial Sequence
  Oligonucleotide
ggatccatgcccatggtgtgcagtcctgctcgtttgc

SEQ ID NO: 26 - DNA, 89 bp, Artificial Sequence
  Oligonucleotide
ggactggaaggctcaatcgatcccggggcagaaaggcccacccgaaggtgagccagtgtgatgatcatcaggcattgagaatttcgtcg

SEQ ID NO: 27 - DNA, 89 bp, Artificial Sequence
  Oligonucleotide
gcacgaaattctcaatgcctgatgatcatcacactggctcaccttcgggtgggcctttctgccccgggatcgattgagccttccagtcc

SEQ ID NO: 28 - DNA, 28 bp, Artificial Sequence
  Oligonucleotide
ggatccttaccagtaatgctccgctgtc

SEQ ID NO: 29 - DNA, 38 bp, Artificial Sequence
  Oligonucleotide
cccaagctttgacttctatgacctggctggaaccgcag

SEQ ID NO: 30 - DNA, 33 bp, Artificial Sequence
  Oligonucleotide
ggaattcttaaaacaccggggtttcacgatagg

SEQ ID NO: 31 - DNA, 39 bp, Artificial Sequence
  Oligonucleotide
cccaagctttgacttctatggatcagattccgattcgtg

SEQ ID NO: 32 - DNA, 32 bp, Artificial Sequence
  Oligonucleotide
ggaattcttaacgcgcaccacgcgccgcaacc

SEQ ID NO: 33 - DNA, 40 bp, Artificial Sequence
  Oligonucleotide
cccaagctttgatgagtcaggcgctaaaaaatttactgac

SEQ ID NO: 34 - DNA, 35 bp, Artificial Sequence
  Oligonucleotide
ggaattcttaattgtgattacgcatcatttcttcc

SEQ ID NO: 35 - DNA, 36 bp, Artificial Sequence
  Oligonucleotide
cccaagctttgacttctatgattaagagttttaatg

SEQ ID NO: 36 - DNA, 34 bp, Artificial Sequence
  Oligonucleotide
ggaattcttatttattgcctgcaactaaagctgc

SEQ ID NO: 37 - DNA, 51 bp, Artificial
  Oligonucleotide
cccgctcttcaaagctttgacttctatgtatagattactaataatcaatcc

SEQ ID NO: 38 - DNA, 37 bp, Artificial Sequence
  Oligonucleotide
ggaattcttatttgtattccttagctttttcttctcc

(19) United States
(12) Patent Application Publication
(10) Pub. No.: US 2011/0151530 A1
(43) Pub. Date: Jun. 23, 2011

(54) ENZYMATIC PRODUCTION OF 2-HYDROXY-ISOBUTYRATE (2-HIBA)

(75) Inventors: Philippe Soucaille, Deyme (FR); Cédric Boisart, Gerzat (FR)

(73) Assignee: METABOLIC EXPLORER, Saint Beauzire (FR)

(21) Appl. No.: 13/060,636

(22) PCT Filed: Aug. 25, 2009
(86) PCT No.: PCT/EP2009/060942

§ 371 Date: Feb. 24, 2011

(30) Foreign Application Priority Data
Aug. 25, 2008 (EP) PCT/EP2008/061091

(51) Int. Cl. C12P 7/42; C12N 1/00; C12N 1/21

(52) U.S. Cl. 435/146; 435/243; 435/252.3

(57) ABSTRACT

The present invention concerns a new method for the biological preparation of 2-hydroxy-isobutyrate (2-HIBA), including a fermentation method with microorganisms modified to favour production of 2-HIBA from renewable resources. The invention also concerns the modified microorganisms used in such fermentation method.

The present invention concerns a new method for the biological preparation of 2-hydroxy-isobutyrate (2-HIBA), including a fermentation method with microorganisms modified to favour production of 2-HIBA from renewable resources. The invention also concerns the modified microorganisms used in such fermentation method.

### BACKGROUND

2-hydroxyisobutyric acid (2-HIBA) is also called 2-hydroxyisobutyrate, 2-Hydroxy-2-methylpropionic acid; and 2-methyllactic acid. CAS Registration number is 594-61-6.

2-hydroxyisobutyric acid is a precursor of the three compounds: 2,3-dihydroxy-methylproprionate, 2-propanol and methacrylate.

Methacrylate, also called methacrylic acid, is a large volume chemical (3.2 millions tons were produced in 2005) compound widely used in resins, plastics, rubber, and denture material. It is currently produced from highly toxic chemicals like hydrogen cyanide, formaldehyde or methacrolein. It is therefore important to develop an environmentally friendly and cost-efficient method to produce methacrylic acid.

### PRIOR ART

Up to now, the enzymatic production of 2-HIBA was performed as shown below:

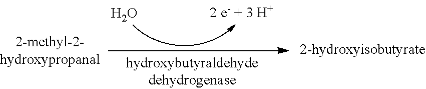

For reference see (Lopes Ferreira N, Labbe D, Monot F, Fayolle-Guichard F, Greer C W. Microbiology (2006) 152: 1361-74.)

The article from Rohwerter et al. entitled “The Alkyl tert-Butyl Ether Intermediate 2-Hydroxyisobutyrate is degraded via a novel cobalamin-dependant mutase pathway” describes the degradation pathway of methyl and ethyl tert-butyl ether. The authors focus on the degradation of 2-HIBA, an intermediate in the pathway. Results show that 2-HIBA is degraded by the small subunit of isobutyryl-coA mutase.

WO 2007/110394 from Rohwerter et al. describes a method for the enzymatic conversion of 3-hydroxybutyric acid into 2-hydroxyisobutyric acid with a microorganism expressing a mutase enzymatic activity. The enzymatic conversion is done either with enzymes extracts obtain from the microorganism (example 2) or by culturing the microorganism on a medium comprising the 3-hydroxybutyric acid as the substrate for the enzymatic conversion (example 1). WO 2007/110394 does not disclose a fermentation method where the 2-hydroxyisobutyrate is obtained by conversion of a simple source of carbon, such as glucose, in a microorganism. In the method disclosed in WO 2007/110394, the starting material is 3-hydroxybutyric acid, which may be of any origin, and particularly obtained by conventional chemical synthesis.

U.S. Pat. No. 7,262,037 teaches a method for the production of D-(3)-hydroxybutyric acid by fermentation of recombinant Escherichia coli; FIG. 4 shows the metabolic pathway for the production from glucose to D-(3)-hydroxybutyric acid, via the metabolites acetyl-coA and 3-hydroxybutyryl-coA. Genes phaA and phaB from Ralstonia eutropha are introduced into a recombinant E. coli, and the encoded enzymes perform the first steps of modifications of acetyl-coA. U.S. Pat. No. 7,262,037 does not disclose how to obtain 2-HIBA from a source of carbon such as glucose.

Combining the teaching of WO 2007/110394 and U.S. Pat. No. 7,262,037 would lead to a complex method needing two different microorganisms, a first microorganism for the conversion of glucose into 3-hydroxybutyric acid and a second microorganism for the enzymatic conversion of the 3-hydroxybutyric acid into 2-HIBA.

The process of the present invention with the fermentative preparation of 2-HIBA by culturing a microorganism on renewable material abundant and inexpensive, provides a solution to the problems of the chemical process as well as to the complexity of the enzymatic conversions disclosed in the art.

### GENERAL DESCRIPTION OF THE INVENTION

The present invention is related to a method for the preparation of 2-hydroxyisobutyric acid (2-HIBA) comprising the successive steps allowing conversion of acetyl-CoA into 2-hydroxyisobutyric acid, said successive steps consisting in:
a) converting acetyl-CoA into 3-hydroxybutyryl-CoA
b) converting the 3-hydroxybutyryl-CoA previously obtained in step a) into 2-hydroxyisobutyryl-CoA, and
c) converting 2-hydroxyisobutyryl-CoA into 2-hydroxyisobutyric acid wherein conversions in steps a, b and c are enzymatic conversions.

In a preferred embodiment, the enzymatic conversion are performed in a single microorganism, wherein the microorganism is modified to express the enzymatic activities necessary for the successive steps a), b) and c).

More preferably, the method of the invention is a method for the fermentative production of 2-hydroxyisobutyric acid (2-HIBA) by conversion of a simple source of carbon into 2-HIBA, comprising the steps of:
culturing the microorganism modified to express the enzymatic activities necessary for the successive steps a), b) and c) in an appropriate culture medium comprising a simple source of carbon, and
recovering the 2-HIBA from the culture medium.

Step a) is advantageously performed with the combination of two enzymes, the first enzyme a1) having an acetoacetyl-CoA thiolase or acetyl-CoA acetyltransferase activity and the second enzyme a2) having a 3-hydroxybutyryl-CoA dehydrogenase activity.

Step b) is advantageously performed with an enzymatic system having a hydroxyisobutyryl-CoA mutase activity.

Advantageously, step c) is performed by transfer of CoA on a substrate with an enzyme having a CoA transferase activity or acyl-CoA thioesterase activity, or with the combination of two enzymes, the first enzyme c1) having a phosphotransacylase activity and the second enzyme c2) having an acid-kinase activity.

Advantageously, the enzymatic activities necessary for the successive steps a), b) and c) are encoded by exogenous genes.

The modified microorganisms are also part of the present invention.

### DETAILED DESCRIPTION OF THE INVENTION

The present invention is related to a method for the preparation of 2-hydroxyisobutyric acid (2-HIBA) comprising the successive steps allowing conversion of acetyl-CoA into 2-hydroxyisobutyric acid, said successive steps consisting in

a) converting acetyl-CoA into 3-hydroxybutyryl-CoA

b) converting 3-hydroxybutyryl-CoA into 2-hydroxyisobutyryl-CoA, and

c) converting 2-hydroxyisobutyryl-CoA into 2-hydroxyisobutyric acid.

The primary substrate, acetyl-CoA, is an important molecule in metabolism. Acetyl-coA is a product of the carbohydrate metabolism. Its main use is to convey the carbon atoms within the acetyl group to the citric acid cycle to be oxidized for energy production. Acetyl-CoA is produced during the second step of aerobic cellular respiration.

The steps a), b) and c) are enzymatic conversions of the specified compounds. The terms “enzyme activity” and “enzymatic activity” are used interchangeably and refer to the ability of an enzyme or a combination of enzymes to catalyse a specific chemical reaction.

The preparation of 2-HIBA according to the invention can be performed in a liquid reaction medium, with exogenous addition of the substrate acetyl-coA, or in one or more microorganisms used as “mini-factories”, preferentially in one microorganism, said microorganism being able to synthesize acetyl-coA from any source of carbon.

In the description of the present invention, enzymatic activities are also designated by reference to the genes coding for the enzymes having such activity. Genes and proteins are generally identified using the denominations of genes from Escherichia coli, Clostridium acetobutylicum, Ralstonia eutropha, Aquincola tertiaricarbonis and Methylibium petroleiphilum. However, use of these denominations has a more general meaning according to the invention and covers all the corresponding genes and proteins in other organisms, more particularly microorganisms, functional homologues, functional variants and functional fragments of said genes and proteins.

Using the references of the IUBMB Enzyme Nomenclature for known enzymatic activities, those skilled in the art are able to determine the same enzymatic activities in other organisms, bacterial strains, yeasts, fungi, etc. This routine work is advantageously done using consensus sequences that can be determined by carrying out sequence alignments with proteins derived from other microorganisms.

Methods for the determination of the percentage of homology between two protein sequences are known from the man skilled in the art. For example, it can be made after alignment of the sequences by using the software CLUSTALW available on the website http://www.ebi.ac.uk/clustalw/ with the default parameters indicated on the website. From the alignment, calculation of the percentage of identity can be made easily by recording the number of identical residues at the same position compared to the total number of residues. Alternatively, automatic calculation can be made by using for example the BLAST programs available on the website http://www.ncbi.nlm.nih.gov/BLAST/ with the default parameters indicated on the website.

PFAM (protein families database of alignments and hidden Markov models; http://www.sanger.ac.uk/Software/Pfam/) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.gov/COG/ are obtained by comparing protein sequences from 66 fully sequenced genomes representing 30 major phylogenic lines. Each COG is defined from at least three lines, which permits the identification of former conserved domains.

A protein sharing homology with the cited protein may be obtained from other microorganisms or may be a variant or a functional fragment of a natural protein.

The term “functional variant or functional fragment” means that the amino-acid sequence of the polypeptide may not be strictly limited to the sequence observed in nature, but may contain additional amino-acids. The term “functional fragment” means that the sequence of the polypeptide may include less amino-acid than the original sequence but still enough amino-acids to confer the enzymatic activity of the original sequence of reference. It is well known in the art that a polypeptide can be modified by substitution, insertion, deletion and/or addition of one or more amino-acids while retaining its enzymatic activity. For example, substitution of one amino-acid at a given position by a chemically equivalent amino-acid that does not affect the functional properties of a protein are common. For the purpose of the present invention, substitutions are defined as exchanges within one of the following groups:
Small aliphatic, non-polar or slightly polar residues: Ala, Ser, Thr, Pro, Gly
Polar, negatively charged residues and their amides: Asp, Asn, Glu, Gln
Polar, positively charged residues: His, Arg, Lys
Large aliphatic, non-polar residues: Met, Leu, Ile, Val, Cys
Large aromatic residues: Phe, Tyr, Trp.

Changes that result in the substitution of one negatively charged residue for another (such as glutamic acid for aspartic acid) or one positively charged residue for another (such as lysine for arginine) can be expected to produce a functionally equivalent product.

The positions where the amino-acids are modified and the number of amino-acids subject to modification in the amino-acid sequence are not particularly limited. The man skilled in the art is able to recognize the modifications that can be introduced without affecting the activity of the protein. For example, modifications in the N- or C-terminal portion of a protein may be expected not to alter the activity of a protein under certain circumstances.

The term “variant” refers to polypeptides submitted to modifications such as defined above while still retaining the original enzymatic activity.

The terms “encoding” or “coding” refer to the process by which a polynucleotide, through the mechanisms of transcription and translation, produces an amino-acid sequence. This process is allowed by the genetic code, which is the relation between the sequence of bases in DNA and the sequence of amino-acids in proteins. One major feature of the genetic code is to be degenerate, meaning that one amino-acid can be coded by more than one triplet of bases (one “codon”). The direct consequence is that the same amino-acid sequence can be encoded by different polynucleotides. It is well known from the man skilled in the art that the use of codons can vary according to the organisms. Among the codons coding for the same amino-acid, some can be used preferentially by a given microorganism. It can thus be of interest to design a polynucleotide adapted to the codon usage of a particular microorganism in order to optimize the expression of the corresponding protein in this organism.

In particular, if the host microorganism is E. coli, genes sequences from other microorganisms such as R. eutropha may be recoded with preferred codons and synthetic genes would be prepared in order to be expressed correctly in E. coli (see examples).

Step a

Conversion of acetyl-coA into 3-hydroxybutyryl-coA is advantageously obtained with the combination of two enzymes:
the first enzyme a1) having an acetoacetyl-CoA thiolase activity or acetyl-CoA acetyltransferase activity (EC 2.3.1.), and
the second enzyme a2) having an 3-hydroxybutyryl-CoA dehydrogenase activity (EC 1.1.1.157).

In a preferred embodiment of the invention, the first enzyme a1) is a gene product encoded by genes selected among the group consisting of:
atoB of E. coli,
thlA of C. acetobutylicum and
phaA of R. eutropha.

The three proteins encoded by genes atoB of E. coli, thlA of C. acetobutylicum and phaA of R. eutropha, all showing the same enzymatic activity, show in their sequences an identity percentage of at least 61%.

Therefore, according to the invention, “a polypeptide having an acetoacetyl-CoA thiolase activity or acetyl-CoA acetyl transferase activity” designates all polypeptides having at least 60% of homology with the sequence of atoB from E. coli, preferentially at least 70% of homology, and more preferentially at least 80% of homology.

In another preferred embodiment of the invention, the second enzyme a2) is a gene product encoded by genes selected among the group consisting of:
hbd of C. acetobutylicum and
phaB of R. eutropha.

Step b

The conversion of 3-hydroxybutyryl-CoA into 2-hydroxyisobutyryl-CoA is preferably obtained with an enzymatic system having a hydroxyisobutyryl-CoA mutase activity.

This activity requires a source of cobalamide coenzyme such as Vitamin B₁₂ that has to be added to the culture medium.

Preferred genes encoding a hydroxyisobutyryl-CoA mutase are icmA and icmB from A. tertiaricarbonis, M. petroleiphilum or from Streptomyces spp.

These hydroxyisobutyryl-CoA mutase enzymes show at least 40% of homology in their amino acid sequences. Therefore, the meaning of “hydroxyisobutyryl-CoA mutase activity” designates all polypeptides having this activity and sharing at least 40% of identity in their amino acid sequences with IcmA and IcmB proteins from Streptomyces spp.

It was shown by the inventors that the activity of the mutase, which is a cobalamide dependent mutase, may be increased by overexpressing the fldA-fpr activation system. Such system is disclosed in the art for SAM-radical enzymes (WO 2007/047680) and the skilled person knows how to overexpress said system.

It was further demonstrated by the inventors that the activity of the mutase was improved in the absence of oxygen in the assay. In one embodiment of the invention, step b) is performed under anaerobic or micro-aerobic conditions.

Step c

In a specific embodiment of the invention, the conversion of 2-hydroxyisobutyryl-CoA into 2-hydroxyisobutyric acid is obtained by transfer of CoA on a substrate with an enzyme having a CoA transferase activity (EC 2.8.3).

Preferentially, the substrates are acetate and 2-hydroxyisobutyryl-CoA and the enzyme has an acetyl-CoA transferase activity (EC 2.3.1.).

In another embodiment of the invention, the conversion of 2-hydroxyisobutyryl-CoA into 2-hydroxyisobutyric acid is obtained by transfer of CoA on a substrate with an enzyme having an acyl-CoA thioesterase activity (EC 3.1.2).

Preferentially, the substrate is 2-hydroxyisobutyryl-CoA and the enzyme has an 2-hydroxyisobutyryl-CoA thioesterase activity, and is a gene product encoded by genes selected among the group consisting of:
tesB of E. coli and
ybgC of H. influenzae.

In another embodiment of the invention, the conversion of 2-hydroxyisobutyryl-CoA into 2-hydroxyisobutyric acid is obtained with the combination of two enzymes:
the first enzyme c1) having a phosphotransacylase activity and
the second enzyme c2) having aa acid-kinase activity.

In a specific aspect of the invention, the enzyme c1) has a phosphate hydroxyisobutyryltransferase activity, and is in particular a gene product encoded by the gene ptb of C. acetobutylicum.

In a specific aspect of the invention, the enzyme c2) is a hydroxyisobutyrate kinase, and is in particular a gene product encoded by the gene buk of C. acetobutylicum.

Microorganisms

In a preferred aspect of the invention, the primary substrate acetyl-CoA is obtained by bioconversion of any source of carbon in a microorganism. This bioconversion happens during the second step of aerobic cellular respiration, where the carbon substrate is converted into energy.

The term ‘carbon source’ or ‘carbon substrate’ or ‘source of carbon’ according to the present invention denotes any source of carbon that can be used by those skilled in the art to support the normal growth of a micro-organism, including hexoses (such as glucose, galactose or lactose), pentoses, monosaccharides, disaccharides, oligosaccharides (such as sucrose, cellobiose or maltose), molasses, starch or its derivatives, hemicelluloses, glycerol and combinations thereof. An especially preferred simple carbon source is glucose. Another preferred simple carbon source is sucrose.

In a preferred embodiment of the invention, steps a), b) and c) are performed by a microorganism expressing the genes coding for the enzymes having the enzymatic activities necessary for the conversions of said steps a), b) and c).

Preferentially, steps a), b) and c) are performed by the same microorganism that express all gene products, necessary for the realisation of enzymatic reactions, such as described previously.

More preferentially, it is the same microorganism that provides for the bioconversion of glucose into acetyl-CoA before hosting the enzymatic reactions a), b) and c). Therefore, the conversion of glucose into 2-HIBA is performed in a single microorganism.

The invention is also relative to a microorganism for the preparation of 2-hydroxyisobutyric acid, wherein said microorganism expresses the genes coding for the enzymes having the enzymatic activities necessary for the conversions of said steps a), b) and c) as defined previously.

The invention therefore provides for a microorganism modified for an improved production of 2-HIBA, wherein said microorganism is modified to express the genes coding for enzymes allowing conversion of acetyl-CoA into 2-hydroxyisobutyric acid by the successive steps a), b) and c) consisting in:
a) converting acetyl-CoA into 3-hydroxybutyryl-CoA
b) converting 3-hydroxybutyryl-CoA previously obtained into 2-hydroxyisobutyryl-CoA, and
c) converting 2-hydroxyisobutyryl-CoA into 2-hydroxyisobutyric acid.

The enzymes allowing such enzymatic activities and the gene coding for such enzymatic activities are disclosed above and below.

The microorganism of the invention is a “microorganism modified for an improved production of 2-HIBA” in which pathways to favour the production of 2-HIBA by conversion of a simple source of carbon have been modified. The microorganism modified for such improved production produces more of the desired biochemical than a native, unmodified microorganism.

According to the invention, the term “microorganism” designates a bacterium, yeast or a fungus. Preferentially, the microorganism is selected among Enterobacteriaceae, Clostridiaceae, Bacillaceae, Streptomycetaceae and Corynebacteriaceae. More preferentially the microorganism is a species of Escherichia, Clostridium, Bacillus, Klebsiella, Pantoea, Salmonella or Corynebacterium. Even more preferentially the microorganism is either the species Escherichia coli or Corynebacterium glutamicum or Clostridium acetobutylicum or Bacillus subtilis.

A microorganism can express exogenous genes if these genes are introduced into the microorganism with all the elements allowing their expression in the host microorganism. Transforming microorganisms with exogenous DNA is a routine task for the man skilled in the art.

Exogenous genes can be integrated into the host genome, or be expressed extrachromosomally by plasmids or vectors. Different types of plasmids are known by the man skilled in the art, which differ with respect to their origin of replication and their copy number in the cell.

According to the invention and except mentioned otherwise, “overexpressing” a gene or “overexpression” means either:
introducing at least one or more exogenous genes in a microorganism when the enzymatic activity produced by expressing said one or more genes is not present in the non-transformed microorganism, or
increasing the expression of one or more genes presents in a microorganism having the said enzymatic activity.

The skilled artisan knows how to overexpress a gene in a microorganism, particularly with introducing one or more copy of the gene in the microorganism of by modifying the level of expression of a gene under control of a modified promoter.

Important elements for controlling the expression of genes are promoters. In a preferred embodiment of the invention, genes may be expressed using promoters with different strength, which may be inducible. These promoters may be homologous or heterologous. The man skilled in the art knows how to choose the promoters that are the most convenient, for example promoters Ptrc, Ptac, Plac or the lambda promoter a are widely used.

All techniques for transforming the microorganisms, and regulatory elements used for enhancing production of the protein of the invention are well known in the art and available in the literature, including applicant's own patent applications on modification of biosynthesis pathways in various microorganisms, including WO2008/052973, WO2008/052595, WO2008/040387, WO2007/144346, WO2007/141316, WO2007/077041, WO2007/017710, WO2006/082254, WO2006/082252, WO2005/111202, WO2005/073364, WO2005/047498, WO2004/076659, the content of which is incorporated herein by reference.

In most preferred embodiments, the microorganism of the invention is a modified microorganism expressing the following genes:
phaA (R. eutropha), phaB (R. eutropha), icmA (A. tertiaricarbonis, M. petroleiphilum), icmB (A. tertiaricarbonis, M. petroleiphilum), tesB (E. coli); or
phaA (R. eutropha), phaB (R. eutropha), icmA (A. tertiaricarbonis, M. petroleiphilum), icmB (A. tertiaricarbonis, M. petroleiphilum), ybgC (H. influenzae); or
phaA (R. eutropha), phaB (R. eutropha), icmA (A. tertiaricarbonis, M. petroleiphilum), icmB (A. tertiaricarbonis, M. petroleiphilum), ptb (C. acetobutylicum), buk (C. acetobutylicum); or
thlA (C. acetobutylicum), hbd (C. acetobutylicum), icmA (A. tertiaricarbonis, M. petroleiphilum), icmB (A. tertiaricarbonis, M. petroleiphilum), tesB (E. coli); or
thlA (C. acetobutylicum), hbd (C. acetobutylicum), icmA (A. tertiaricarbonis, M. petroleiphilum), icmB (A. tertiaricarbonis, M. petroleiphilum), ybgC (H. influenzae); or
thlA (C. acetobutylicum), hbd (C. acetobutylicum), icmA (A. tertiaricarbonis, M. petroleiphilum), icmB (A. tertiaricarbonis, M. petroleiphilum), ptb (C. acetobutylicum), buk (C. acetobutylicum); or
atoB (E. coli), hbd (C. acetobutylicum) or phaB (R. eutropha), icmA (A. tertiaricarbonis, M. petroleiphilum), icmB (A. tertiaricarbonis, M. petroleiphilum), tesB (E. coli); or
atoB (E. coli), hbd (C. acetobutylicum) or phaB (R. eutropha), icmA (A. tertiaricarbonis, M. petroleiphilum), icmB (A. tertiaricarbonis, M. petroleiphilum), ybgC (H. influenzae); or
atoB (E. coli), hbd (C. acetobutylicum) or phaB (R. eutropha), icmA (A. tertiaricarbonis, M. petroleiphilum), icmB (A. tertiaricarbonis, M. petroleiphilum), ptb (C. acetobutylicum), buk (C. acetobutylicum)

In a particular embodiment, the microorganism of the invention as defined above and below is also modified to produce higher levels of acetyl-CoA.

The flux toward acetyl-CoA may be increased by different means, and in particular:
i) decreasing the activity of the enzyme lactate dehydrogenase by attenuation of the gene ldhA,
ii) decreasing the activity of at least one of the following enzymes:
   phospho-transacetylase, encoded by the pta gene
   acetate kinase, encoded by the ackA gene
   pyruvate oxidase, encoded by the poxB gene by attenuation of the genes,
iii) decreasing the activity of the enzyme isocitrate lyase, encoded by the aceA gene.

An embodiment of the invention also provides a better yield of 2-HIBA production by increasing NADPH availability. This increased availability in the microorganism can be obtained through the attenuation of at least one of the genes selected among the following: pgi encoding the glucose-6-phosphate isomerase or udhA encoding the soluble transhydrogenase. With such genetic modifications, the glucose-6-phosphate will have to enter glycolysis through the pentose phosphate pathway and a maximum of 2 NADPH will be produced per glucose-6-phosphate metabolized.

A microorganism modified for an improved production of 2-HIBA with increased NADPH availability is also part of the invention.

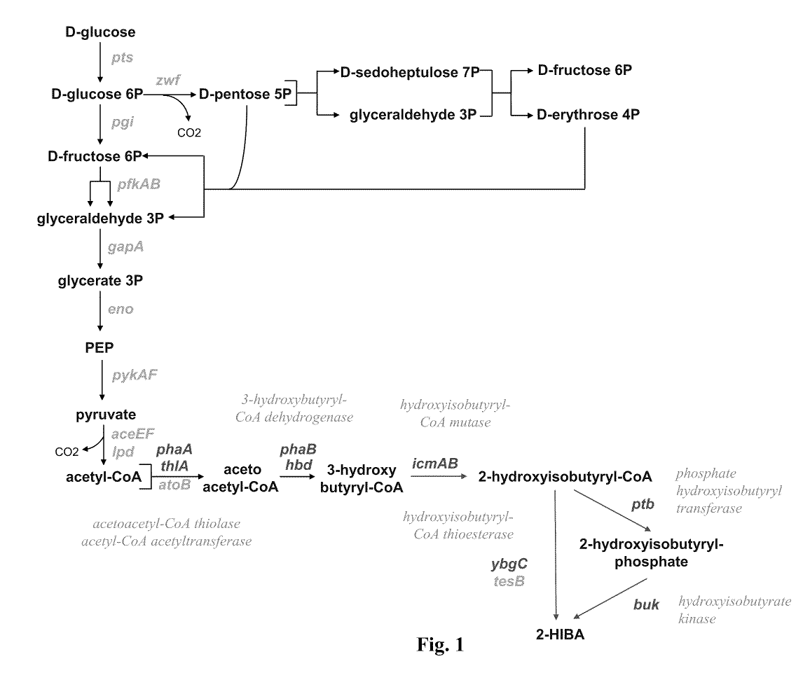

A biosynthetic pathway from glucose to 2-HIBA including steps a), b) and c) is shown on FIG. 1.

Fermentative Production

The present invention also discloses a method for the fermentative production of 2-hydroxyisobutyric acid (2-HIBA) by conversion of a simple source of carbon into 2-HIBA, comprising the steps of:
culturing a microorganism according to the invention, as defined above and below, in an appropriate culture medium comprising a simple source of carbon, and
recovering the 2-hydroxyisobutyric acid (2-HIBA) from the culture medium.

More particularly the invention provides for a method for the fermentative production of 2-hydroxyisobutyric acid (2-HIBA) by conversion of a simple source of carbon into 2-HIBA, comprising the steps of:
culturing a microorganism modified for an improved production of 2-HIBA in an appropriate culture medium comprising a simple source of carbon, and
recovering the 2-hydroxyisobutyric acid (2-HIBA) from the culture medium,
   wherein said microorganism is modified to express the genes coding for enzymes allowing conversion of acetyl-CoA into 2-hydroxyisobutyric acid by the successive steps a), b) and c) consisting in
a) converting acetyl-CoA into 3-hydroxybutyryl-CoA
b) converting 3-hydroxybutyryl-CoA previously obtained into 2-hydroxyisobutyryl-CoA, and
c) converting 2-hydroxyisobutyryl-CoA into 2-hydroxyisobutyric acid.

In preferred embodiments, the microorganism is also modified to produce higher levels of acetyl-CoA. It may also be modified to increase NADPH availability.

All modifications of the microorganism are disclosed above and below.

The fermentation is generally conducted in fermenters with an appropriate culture medium adapted to the microorganism being used, containing at least one simple carbon source, and if necessary co-substrates.

The fermentation may be conducted under aerobic, micro-aerobic or anaerobic conditions. In some embodiments, it was found that culture under micro-aerobic conditions may provide increased yields.

In the production method of the invention, the microorganism is cultured on an appropriate culture medium.

An “appropriate culture medium” means a medium of known molecular composition adapted to the growth of the micro-organism. In particular, said medium contains at least a source of phosphorus and a source of nitrogen. Said appropriate medium is for example a mineral culture medium of known set composition adapted to the bacteria used, containing at least one carbon source. Said appropriate medium may also designate any liquid comprising a source of nitrogen and/or a source of phosphorus, said liquid being added and/or mixed to the source of sucrose. In particular, the mineral growth medium for Enterobacteriaceae can thus be of identical or similar composition to M9 medium (Anderson, 1946), M63 medium (Miller, 1992) or a medium such as defined by Schaefer et al. (1999).

As an example of known culture mediums for E. coli, the culture medium can be of identical or similar composition to an M9 medium (Anderson, 1946, Proc. Natl. Acad. Sci. USA 32:120-128), an M63 medium (Miller, 1992; A Short Course in Bacterial Genetics: A Laboratory Manual and Handbook for Escherichia coli and Related Bacteria, Cold Spring Harbor Laboratory Press, Cold Spring Harbor, N.Y.) or a medium such as defined by Schaefer et al. (1999, Anal. Biochem. 270: 88-96).

As another example of culture medium for C. glutamicum, the culture medium can be of identical or similar composition to BMCG medium (Liebl et al., 1989, Appl. Microbiol. Biotechnol. 32: 205-210) or to a medium such as described by Riedel et al. (2001, J. Mol. Microbiol. Biotechnol. 3: 573-583).

Those skilled in the art are able to define the culture conditions for the microorganisms according to the invention. In particular the bacteria are fermented at a temperature between 20° C. and 55° C., preferentially between 25° C. and 40° C., and more specifically about 30° C. for C. glutamicum and about 37° C. for E. coli.

The carbon source “glucose” can be replaced in this medium by any other carbon source, in particular by sucrose or any sucrose-containing carbon source such as sugarcane juice or sugar beet juice.

A “carbon source” or “carbon substrate” means any carbon source capable of being metabolized by a microorganism wherein the substrate contains at least one carbon atom.

Preferably, the carbon source is selected among the group consisting of glucose, sucrose, mono- or oligosaccharides, starch or its derivatives or glycerol and mixtures thereof.

Indeed the microorganisms used in the method of the present invention can also be modified to be able to grow on specific carbon sources when the non modified microorganism cannot grow on the same source of carbon, or grow at to low rates. These modifications may be necessary when the source of carbon is a byproduct of biomass degradation such as by-products of sugarcane including; filter cake from clarification of raw juice and different kind of molasses.

Recovering the 2-hydroxyisobutyric acid from the culture medium is a routine task for a man skilled in the art.

In one aspect of the invention, the recovered 2-hydroxyisobutyric acid (2-HIBA) is further purified.

### EXAMPLES

### 1 Example 1

### Construction of a Strain Producing 2-Hydroxyisobutyric Acid: MG1655 pME101-icmAmp-icmBmp-TT07-Plac-phaAre-phaBre-TT02

In order to construct a strain of E. coli MG1655 producing 2-hydroxyisobutyric acid, genes icmA and icmB of Methylibium petroleiphilum coding for the mutase activity will be overexpressed on a plasmid in combination with the phaA and phaB genes from Ralstonia eutropha coding respectively for the acetyl-CoA acetyltransferase and 3-hydroxybutyryl-CoA dehydrogenase activities. Each individual and combined constructions are detailed below.

1.1 Construction of a Plasmid for Overexpression of the Hydroxyisobutyryl-CoA Mutase icmA and icmB of M. petroleiphilum: pME101-icmAmp-icmBmp-TT07 Plasmid

For this purpose synthetic genes of the M. petroleiphilum icmA and icmB hydroxyisobutyryl-CoA mutase genes were prepared by the Geneart company. The codon usage and GC content of the genes was adapted to E. coli according to the supplier matrix. Expression of the synthetic genes, organized in operon, was driven by a constitutive Ptrc promoter. Transcriptional terminator was added downstream of the genes. The construct was cloned into supplier's pM vectors and verified by sequencing. The construct may therefore be cloned in the pME101 vector (This plasmid is derived from plasmid pCL1920 (Lerner & Inouye, 1990, NAR 18, 15 p 4631)) if necessary before transforming an E. coli strain.

Ptrc01-icmAmp-icmBmp-TT07 is composed of:

SEQ ID N^{o}1:
restriction sites (BamHI, HindIII, EcoRV):
ggatccatgcaagcttatgcgatatc
SEQ ID N^{o}2: Ptrc01 promoter:
gagctgttgacaattaatcatccggctcgtataatgtgtggaataagga
ggtatatc
SEQ ID N^{o}3:
icmAmp gene sequence optimized for E. coli
(YP_001023546.):
SEQ ID N^{o}4: intergenic sequence:
gaataaggaggtatatt
SEQ ID N^{o}5:
icmBmp gene sequence optimized for E. coli
(YP_001023543.):
SEQ ID N^{o}6: terminator sequence T7Te (ref:
Harrington K. J., Laughlin R. B. and Liang S. Proc
Natl Acad Sci U S A. 2001 Apr. 24;98(9):5019-24.):
ctggctcaccttcgggtgggcctttctg
SEQ ID N^{o}7:
restriction sites (SmaI, BamHI, EcoRI):
ccccgggatgcggatccatgcgaattc

For the expression from a low copy vector the plasmid pME101 can be constructed as follows. The plasmid pCL1920 is PCR amplified using the oligonucleotides PME101F and PME101R and the BstZ171-XmnI fragment from the vector pTRC99A harboring the lad gene and the Ptrc promoter is inserted into the amplified vector. The resulting vector and the vector harboring the icmAmp and icmBmp genes can be restricted by NcoI and BamHI and the icmAmp and icmBmp containing fragment can be cloned into the vector pME101: the resulting plasmid is named pME101-icmAmp-icmBmp-TT07

PME101F (SEQ ID NO 8):
ccgacagtaa gacgggtaag cctg
PME101R (SEQ ID NO 9):
agcttagtaa agccctcgct ag

1.2 Construction of a Plasmid for Overexpression of the Acetyl-CoA Acetyltransferase phaA and the 3-Hydroxybutyryl-CoA Dehydrogenase phaB of R. eutropha: pME101-phaAre-phaBre-TT02 Plasmid

As previously described, for this purpose synthetic genes of the R. eutropha phaA acetyl-CoA acetyltransferase and phaB 3-hydroxybutyryl-CoA dehydrogenase genes were prepared by the Geneart company. Expression of the synthetic genes, organized in operon, was driven by a constitutive Plac promoter. Transcriptional terminator was added downstream of the genes. The construct was cloned into supplier's pM vectors and verified by sequencing. The construct may therefore be cloned the pME101 vector if necessary before transforming an E. coli strain.

Plac-phaAre-phaBre-TT02 is composed of:

SEQ ID N^{o}10:
restriction sites (BamHI, HindIII, SmaI)
ggatccatgcaagcttatgccccggg
SEQ ID N^{o}11: Plac promoter:
aagctcactcattaggcaccccaggctttacactttatgcttccggctc
gtatgttgtgtggaattgtgagcggataacaatttcacacaggactaca
ca
SEQ ID N^{o}12:
phaAre gene sequence optimized for E. coli
(YP_725941.)
SEQ ID N^{o}13:
intergenic sequence:
ggaaggggttttccggggccgcgcgcggttggcgcggacccggcgacga
taacgaagccaatcaaggagtggac
SEQ ID N^{o}14:
phaBre gene sequence optimized for E. coli
(YP_725842.)
SEQ ID N^{o}15:
terminator sequence rrnB t1 (ref: Harrington
K. J., Laughlin R. B. and Liang S. Proc Natl
Acad Sci U S A. 2001 Apr. 24;98(9):5019-24.):
catcaaataaaacgaaaggctcagtcgaaagactgggcctttcgtttta
tctgtt
SEQ ID N^{o}16:
restriction sites (BstZ17I, BamHI, EcoRI):
gtatactgcaggatccatgcgaattc

The pME101 vector and the vector harboring the phaAre and phaBre genes can be restricted by BsrBI and BamHI and the phaAre and phaBre containing fragment can be cloned into the vector pME101, the resulting plasmid is named pME101-phaAre-phaBre-TT02.
1.3 Construction of a Plasmid for Overexpression of the Hydroxyisobutyryl-CoA Mutase icmA and icmB of M. petroleiphilum and the Acetyl-CoA Acetyltransferase phaA and the 3-hydroxybutyryl-CoA Dehydrogenase phaB of ralstonia eutropha: pME101-icmAmp-icmBmp-TT07-Plac-phaAre-phaBre-TT02 Plasmid

In order to co-expressed the icmA, icmB, phaA and phaB genes in an E. coli strain, the pME101-icmAmp-icmBmp-TTO7 vector and the vector harboring the pME101-phaAre-phaBre-TT02 genes can be restricted by SmaI and EcoRI and the phaAre and phaBre containing fragment can be cloned into the vector pME101-icmAmp-icmBmp-TT07, the resulting plasmid is named pME101-icmAmp-icmBmp-TT07-Plac-phaAre-phaBre-TT02.

1.4 Construction of Strains MG1655 pME101-phaAre-phaBre-TT02 and MG1655 pME101-icmAmp-icmBmp-TT07-Plac-phaAre-phaBre-TT02

The pME101-phaAre-phaBre-TT02 and the pME101-icmAmp-icmBmp-TT07-Plac-phaAre-phaBre-TT02 plasmids described above are introduced in the strain MG1655. The first strain obtained MG1655 pME101-phaAre-phaBre-TT02 is named HI0008, the second strain obtained MG1655 pME101-icmAmp-icmBmp-TT07-Plac-phaAre-phaBre-TT02 is named HI0012.

### 2 Example 2

### Construction of Strain Producing 2-Hydroxyisobutyric Acid: MG1655 pUC18-Ptrc01-icmAmp-icmBmp-TT07-Plac-phaAre-phaBre-TT02

In order to obtain higher expression of the icmA and icmB genes of Methylibium petroleiphilum coding for the mutase activity and the phaA and phaB genes from Ralstonia eutropha coding respectively for the acetyl-CoA acetyltransferase and 3-hydroxybutyryl-CoA dehydrogenase activities, we introduce these genes in a high-copy-number pUC18 plasmid.

2.1 Construction of a Plasmid for Overexpression of the Hydroxyisobutyryl-CoA Mutase icmA and icmB of Methylibium petroleiphilum: pUC18-Ptrc01-icmAmp-icmBmp-TT07 Plasmid

The vector harboring the icmAmp and icmBmp genes (pM-Ptrc01-icmAmp-icmBmp-TT07 Geneart's pM vector) is restricted by SmaI and HindIII and the icmAmp and icmBmp containing fragment is cloned into the vector pUC18 restricted by the same restriction enzymes, the resulting plasmid is named pUC18-Ptrc01-icmAmp-icmBmp-TT07.

2.2 Construction of a Plasmid for Overexpression of the Hydroxyisobutyryl-CoA Mutase icmA and icmB of Methylibium petroleiphilum and Overexpression of the Acetyl-CoA Acetyltransferase phaA and the 3-Hydroxybutyryl-CoA Dehydrogenase phaB of Ralstonia eutropha: pUC18-Ptrc01-icmAmp-icmBmp-TT07-Plat-phaAre-phaBre-TT02 Plasmid

The vector harboring the phaAre and phaBre genes (pM-Plac-phaAre-phaBre-TT02 Geneart's pM vector) is restricted by SmaI and EcoRI and the phaAre and phaBre containing fragment is cloned into the vector pUC18-Ptrc01-icmAmp-icmBmp-TT07 restricted by the same restriction enzymes, the resulting plasmid is named pUC18-Ptrc01-icmAmp-icmBmp-TT07-Plac-phaAre-phaBre-TT02.

2.3 Construction of Strain MG1655 pUC18-Ptrc01-icmAmp-icmBmp-TT07-Plac-phaAre-phaBre-TT02

The pUC18-Ptrc01-icmAmp-icmBmp-TT07-Plac-phaAre-phaBre-TT02 plasmid described above is introduced in the strain MG1655. The strain obtained MG1655 pUC18-Ptrc01-icmAmp-icmBmp-TT07-Plac-phaAre-phaBre-TT02 is named HI0060.

### 3 Example 3

### Construction of Strain with Increased NADPH Availability: MG1655 Δpgi ΔudhA pUC18-Ptrc01-icmAmp-icmBmp-TT07-Plac-phaAre-phaBre-TT02

In order to increase NADPH availability to optimize 2-hydroxyisobutyric acid production, we introduce the plasmids described above in a strain E. coli MG1655 in which the pgi (encoding glucose-6-phosphate isomerase) and udhA (encoding the soluble transhydrogenase) genes have been deleted.

### 3.1 Construction of Strain MG1655 Δpgi::Cm

To delete the pgi gene the homologous recombination strategy described by Datsenko & Wanner (2000) is used. This strategy allows the insertion of a chloramphenicol or a kanamycin resistance cassette, while deleting most of the genes concerned. For this purpose the following oligonucleotides are used:

DpgiF (SEQ ID NO 17):
ccaacgcagaccgctgcctggcaggcactacagaaacacttcgatgaaa
tgaaagacgttacgatcgccgatctttttgcTGTAGGCTGGAGCTGCTT
CG

with
a region (lower case) homologous to the sequence (4231352-4231431) of the gene pgi (reference sequence on the website http://genolist.pasteur.fr/Colibri/),
a region (upper case) for the amplification of the chloramphenicol resistance cassette (reference sequence in Datsenko, K. A. & Wanner, B. L., 2000, PNAS, 97: 6640-6645),

DpgiR (SEQ ID NO 18):
gcgccacgctttatagcggttaatcagaccattggtcgagctatcgtgg
ctgctgatttctttatcatctttcagctctgCATATGAATATCCTCCTT
AG

with
a region (lower case) homologous to the sequence (4232980-4232901) of the gene pgi (reference sequence on the website http://genolist.pasteur.fr/Colibri/),
a region (upper case) for the amplification of the chloramphenicol resistance cassette (reference sequence in Datsenko, K. A. & Wanner, B. L., 2000, PNAS, 97: 6640-6645).

The oligonucleotides DpgiF and DpgiR are used to amplify the chloramphenicol resistance cassette from the plasmid pKD3. The PCR product obtained is then introduced by electroporation into the strain MG1655 (pKD46). The chloramphenicol resistant transformants are then selected and the insertion of the resistance cassette is verified by a PCR analysis with the oligonucleotides pgiF and pgiR defined below. The strain retained is designated MG1655 Δpgi::Cm

pgiF (SEQ ID NO 19): gcgggg
cggttgtcaacgatggggtcatgc.
(homologous to the sequence
from 4231138 to 4231167)
pgiR (SEQ ID NO 20): cggtat
gatttccgttaaattacagacaag.
(homologous to the sequence
from 4233220 to 4233191)

3.2 Construction of Strain MG1655 Δpgi ΔudhA

The udhA gene is deleted in the MG1655 Δpgi::Cm strain by transduction. The MG1655 ΔudhA::Km strain is first constructed using the same method as previously described with the following oligonucleotides:

DudhAF (SEQ ID NO 21):
CCCAGAATCTCTTTTGTTTCCCGATGGAACAAAATTTTCAGCGTGCCCA
CGTTCATGCCGACGATTTGTGCGCGTGCCAGTGTAGGCTGGAGCTGCTT
CG

with
a region (boldface letters) homologous to the sequence (4157588-4157667) of the gene udhA (reference sequence on the website http://genolist.pasteur.fr/Colibri/),
a region (underlined letters) for the amplification of the kanamycin resistance cassette (reference sequence in Datsenko, K. A. & Wanner, B. L., 2000, PNAS, 97: 6640-6645),

DudhAR (SEQ ID NO 22):
GGTGCGCGCGTCGCAGTTATCGAGCGTTATCAAAATGTTGGCGGCGGTT
GCACCCACTGGGGCACCATCCCGTCGAAAGCCATATGAATATCCTCCTT
AG

with
a region (boldface letters) homologous to the sequence (4158729-4158650) of the gene udhA (reference sequence on the website http://genolist.pasteur.fr/Colibri/),
a region (underlined letters) for the amplification of the kanamycin resistance cassette (reference sequence in Datsenko, K. A. & Wanner, B. L., 2000, PNAS, 97: 6640-6645).

The oligonucleotides DudhAF and DudhAR are used to amplify the kanamycin resistance cassette from the plasmid pKD4. The PCR product obtained is then introduced by electroporation into the strain MG1655 (pKD46). The kanamycin resistant transformants are then selected and the insertion of the resistance cassette is verified by a PCR analysis with the oligonucleotides udhAF and udhAR defined below. The strain retained is designated MG1655 ΔudhA::Km

udhAF (SEQ ID NO 23): (homologous to the sequence
from 4157088 to 4157108) GATGCTGGAAGATGGTCACT.
udhAR (SEQ ID NO 24): (homologous to the sequence
from 4159070 to 4159052) gtgaatgaacggtaacgc.

To transfer the deletion ΔudhA::Km, the method of phage P1 transduction is used. The protocol followed is implemented in 2 steps with the preparation of the phage lysate of the strain MG1655 ΔudhA::Km and then transduction into strain MG1655 Δpgi::Cm. The construction of the strain is described above.

Preparation of Phage Lysate P1:
Inoculation with 100 μl of an overnight culture of the strain MG1655 ΔudhA::Km of 10 ml of LB+Km 50 μg/ml+glucose 0.2%+CaCl₂ 5 mM.
Incubation for 30 min at 37° C. with shaking
Addition of 100 μl of phage lysate P1 prepared on the strain MG1655 (about 1.10⁹ phage/ml).
Shaking at 37° C. for 3 hours until all the cells were lysed.

Addition of 200 μl chloroform and vortexing.

Centrifugation for 10 min at 4500 g to eliminate cell debris.
Transfer of supernatant to a sterile tube and addition of 200 μl chloroform.
Storage of lysate at 4° C.

Transduction
Centrifuging for 10 min at 1500 g of 5 ml of an overnight culture of the strain MG1655 Δpgi::Cm in LB medium.
Suspension of the cell pellet in 2.5 ml of 10 mM MgSO₄, 5 mM CaCl₂
Control tubes: 100 μA cells
   100 μl phages P1 of strain MG1655 ΔudhA::Km
Test tube: 100 μl of cells+100 μl of phages P1 of the strain MG1655 ΔudhA::Km.
Incubation for 30 min at 30° C. without shaking
Addition of 100 μl of 1 M sodium citrate in each tube and vortexing.
Addition of 1 ml of LB.
Incubation for 1 hour at 37° C. with shaking
Spreading on dishes LB+Km 50 μg/ml after centrifuging of tubes for 3 min at 7000 rpm.
Incubation at 37° C. overnight.

Verification of the Strain

The kanamycin resistant transformants are then selected and the deletion of the gene ΔudhA::Km is verified by a PCR analysis with the oligonucleotides udhAF and udhAR previously described. The strain retained is designated MG1655 Δpgi::Cm ΔudhA::Km.

The kanamycin and chloramphenicol resistance cassettes can then be eliminated. The plasmid pCP20 carrying FLP recombinase acting at the FRT sites of the kanamycin and the chloramphenicol resistance cassettes is then introduced into the recombinant sites by electroporation. After a series of cultures at 42° C., the loss of the kanamycin and chloramphenicol resistance cassettes is verified by a PCR analysis with the same oligonucleotides as used previously (pgiF/pgiR and udhAF/udhAR). The strain retained is designated MG1655 Δpgi ΔudhA.

3.3 Construction of Strain MG1655 Δpgi ΔudhA pUC18-Ptrc01-icmAmp-icmBmp-TT07-Plac-phaAre-phaBre-TT02
The pUC18-Ptrc01-icmAmp-icmBmp-TT07-Plac-phaAre-phaBre-TT02 plasmid described above is introduced in the strain MG1655 Δpgi ΔudhA. The strain obtained MG1655 Δpgi ΔudhA pUC18-Ptrc01-icmAmp-icmBmp-TT07-Plac-phaAre-phaBre-TT02 is named HI0062.

### 4 Example 4

### Construction of Strain with Increased hydroxyisobutyryl-CoA Mutase Activity: MG1655 (pME101-icmAmp-icmBmp-TT07-Plac-phaAre-phaBre-TT02) (pJB137-fidA-TT07-fpr)

In order to increase hydroxyisobutyryl-CoA mutase activity, we overexpress the genes coding for flavodoxin and flavodoxin reductase.

4.1 Construction of Plasmid for Overexpression of the Flavodoxin fldA and Overexpression of the Flavodoxin Reductase fpr: pJB137-fldA-TT07-fpr

The plasmid pJB137-fldA-TT07-fpr is derived from plasmid pJB 137 (Blatny et al., 1997; Appl Environ Microbiol. 1997 February; 63(2):370-9.).

For the construction of the plasmid pJB137-fldA-TT07-fpr, first, the fldA-TT07 region was amplified by PCR from MG1655 genomic DNA using the following oligonucleotides, BamHI-NcoI-fldA-F-1 and fldA-fpr-R-2 (reference sequence on the website http://ecogen.org/). Then the TT07-fpr region was amplified by PCR from MG1655 genomic DNA using the following oligonucleotides, fldA-fpr-F-3 and fpr-BamHI-R-4 (reference sequence on the website http://ecogen.org/). In a third step, the fldA-TT07-fpr region was PCR amplified by mixing the fldA-TT07 and TT07-fpr PCR products and by using the BamHI-NcoI-fldA-F-1 and fpr-BamHI-R-4 oligonucleotides. Both fldA-fpr-R-2 and fldA-fpr-F-3 oligonucleotides were designed to overlap over their entire sequence and allow connecting the two fragments by fusion PCR. The resulting PCR product was cloned in the pSCB vector (Stratagene), verified by sequencing and the vector named pSCB-fldA-TT07-fpr.

BamHI-NcoI-fldA-F-1
(SEQ ID NO 25)
GGATCCatgcCCATGGTGTGCAGTCCTGCTCGTTTGC

with
a region (upper underlined case) harbouring BamHI site,
a region (lower case) with extra-bases,
a region (upper italic case) harbouring NcoI site,
a region (upper bold case) homologous to the fldA region (from 710804 to 710784).

fldA-fpr-R-2
(SEQ ID NO 26)
ggactggaaggctcaatcgatCCCGGGGCAGAAAGGCCCA
CCCGAAGGTGAGCCAGTGTGATGATCATCAGGCATTGAGAATTTCGTCG

with
a region (upper bold case) homologous to the fldA region (from 710179 to 710156),
a region (upper underlined case) for T7te transcriptional terminator sequence (Harrington K. J., Laughlin R. B. and Liang S. Proc Natl Acad Sci USA. 2001 Apr. 24; 98(9):5019-24.),
a region (upper italic case) harbouring the SmaI site,
a region (lower case) homologous to the fpr region (from 4112583 to 4112563).

fldA-fpr-F-3
(SEQ ID NO 27)
CGACGAAATTCTCAATGCCTGATGATCATCACACTGGCTCACCTTCGGG
TGGGCCTTTCTGCCCCGGGatcgattgagccttccagtcc

with
a region (upper bold case) homologous to the fldA region (from 710179 to 710156),
a region (upper underlined case) for T7te transcriptional terminator sequence (Harrington K. J., Laughlin R. B. and Liang S. Proc Natl Acad Sci USA. 2001 Apr. 24; 98(9):5019-24.),
a region (upper italic case) harbouring the SmaI site,
a region (lower case) homologous to the fpr region (from 4112583 to 4112563).

| | |
|---|---|
| | (SEQ ID NO 28) |
| fpr-BamHI-R-4 | GGATCCttaccagtaatgctccgctgtc |

with
a region (lower case) homologous to the fpr region (from 4111770 to 4111749),
a region (upper italic case) harbouring the BamHI site.

To transfer the genes fldA and fpr in a low copy vector, the vector pSCB-fldA-TT07-fpr was cut with the restriction enzymes NcoI and BamHI and the fldA-TT07-fpr fragment cloned into the NcoI/BamHI sites of the pJB137 vector, resulting in vector pJB137-fldA-TT07-fpr.

4.2 Construction of Strain MG1655 (pME101-icmAmp-icmBmp-TT07-Plac-phaAre-phaBre-TT02) (pJB137-fldA-TT07-fpr)

The pME101-icmAmp-icmBmp-TT07-Plac-phaAre-phaBre-TT02 and the pJB137-fldA-TT07-fpr plasmids described above are introduced in the strain MG1655. The strain obtained MG1655 (pME101-icmAmp-icmBmp-TT07-Plac-phaAre-phaBre-TT02) (pJB137-fldA-TT07-fpr) is named HI0048.

### 5 Example 5

### Purification of icmA and icmB Genes Product of M. petroleiphilum Encoding for Hydroxyisobutyryl-CoA Mutase, and Demonstration that the Genes Product Catalyze the Reaction 3-Hydroxybutyryl-CoA <-> 2-Hydroxyisobutyryl-CoA

In order to demonstrate that the hydroxyisobutyryl-CoA mutase activity catalyze the conversion of 3-hydroxybutyryl-CoA into 2-hydroxyisobutyryl-CoA, we have purified separately the two subunits icmA and icmB, and reconstituted the active multimeric protein in vitro.

5.1 Construction of the Overexpression Plasmids for icmAmp and icmBmp
5.1.1 Construction of the Plasmid pPAL7VB01-icmAmp

To amplify the gene icmA from Methylibium petroleiphilum, a PCR is carried out using supplier's pM vector from GeneArt company (see Example 1) as template and the primers named pPAL7-icmAmp F and pPAL7-icmAmp R.

pPAL7-icmAmp F
(SEQ ID NO 29)
CCCAAGCTTTGACTTCTATGACCTGGCTGGAACCGCAG
pPAL7-icmAmp R
(SEQ ID NO 30)
GGAATTCTTAAAACACCGGGGTTTCACGATAGG

PCR product is digested with HindIII and EcoRI and cloned into the vector pPAL7 (Profinity eXact pPAL7 Vector Biorad) cut by the same restriction enzymes. The resulting plasmid is named pPAL7VB01-icmAmp. The protein overexpressed brings two adding amino acid (Thr-Ser) which correspond to the spacer between the tag used and the protein in order to ensure successful purification.

Vector pPAL7VB01-icmAmp is introduced into E. coli BL21 star (DE3) chemically competent cells, leading to strain BL21 star (DE3) (pPAL7VB01-icmAmp).

5.1.2 Construction of the Plasmid pPAL7VB01-icmBmp

To amplify the gene icmB from Methylibium petroleiphilum, a PCR is carried out using supplier's pM vector from GeneArt company (see Example 1) as template and the primers named pPAL7-icmBmp F and pPAL7-icmBmp R.

pPAL7-icmBmp F
(SEQ ID NO 31)
CCCAAGCTTTGACTTCTATGGATCAGATTCCGATTCGTG
pPAL7-icmBmp R
(SEQ ID NO 32)
GGAATTCTTAACGCGCACCACGCGCCGCAACC

PCR product is digested with HindIII and EcoRI and cloned into the vector pPAL7 (Profinity eXact pPAL7 Vector Biorad) cut by the same restriction enzymes. The resulting plasmid is named pPAL7VB01-icmBmp. The protein overexpressed brings two adding amino acid (Thr-Ser) which correspond to the spacer between the tag used and the protein in order to ensure successful purification.

Vector pPAL7VB01-icmBmp is introduced into E. coli BL21 star (DE3) chemically competent cells, leading to strain BL21 star (DE3) (pPAL7VB01-icmBmp).

### 5.2 Overproduction of the Two Subunits of the Hydroxyisobutyryl-CoA Mutase, IcmAmp and IcmBmp

The overproduction of the proteins IcmAmp and IcmBmp was done in a 2 11 Erlenmeyer flask, using LB broth (Bertani, 1951, J. Bacteriol. 62:293-300) that was supplemented with 2.5 g/l glucose and 100 ppm of ampicillin. An overnight preculture was used to inoculate a 500 ml culture to an OD_{600 nm} of about 0.1. This preculture was carried out in a 500 ml Erlenmeyer flask filled with 50 ml of LB broth that is supplemented with 2.5 g/l glucose and 100 ppm of ampicillin. The culture was first kept on a shaker at 37° C. and 200 rpm until OD_{600 nm} was about 0.5 and then the culture was moved about one hour on a second shaker at 25° C. and 200 rpm until OD_{600 nm} was 0.6-0.8 (about one hour), before the induction with 500 μM IPTG. The culture was kept at 25° C. and 200 rpm until OD_{600 nm} was around 4, and then it was stopped. Cells are centrifuged at 7000 rpm, 5 minutes at 4° C., and then stored at −20° C.

### 5.3 Purification of the Proteins IcmAmp and IcmBmp

### 5.3.1 Step 1: Preparation of Cell-Free Extracts.

About 100 mg of E. coli biomass was resuspended in 15 ml of 100 mM potassium phosphate pH 7.6, and a protease inhibitor cocktail. The cell suspension was sonicated on ice (Bandelin sonoplus, 70 W) in a 50 ml conical tube during 8 cycles of 30 sec with 30 sec intervals. After sonication, cells were incubated for 30 min at room temperature with 5 mM MgCl2 and 1 UI/ml of DNaseI. Cells debris were removed by centrifugation at 12000 g for 30 min at 4° C.

### 5.3.2 Step 2: Affinity Purification

The protein was purified from the crude cell-extract by affinity on a Profinity column (BIORAD, Bio-Scale Mini Profinity exact cartridge 1 ml) according to the protocol recommended by the manufacturer. The crude extract was loaded on a 1 ml Profinity exact cartridge 1 ml equilibrated with 100 mM potassium phosphate pH 7.6. The column was washed with 10 column volumes of the same buffer and incubated overnight with 100 mM potassium phosphate pH 7.6, 100 mM fluoride at 4° C. The protein was eluted from the column with 2 column volumes of 100 mM potassium phosphate pH 7.6. The tag remained tightly bound to the resin and the purified protein was released. The fractions containing the protein were pooled and dialyzed against 100 mM Tris-HCl, 150 mM NaCl and 10% glycerol pH 8.

Protein concentration was measured using the Bradford protein assay.

### 5.4 Hydroxyisobutyryl-CoA Mutase Assay

The hydroxyisobutyryl-CoA assay was carried out with 50 mM potassium phosphate buffer pH 7.4, 5 mM EDTA, 20 μM Coenzyme B12, 1 mM or 10 mM 3-Hydroxybutyryl-CoA, 10% glycerol and about 17 μg of purified IcmAmp and IcmBmp mixed in equimolar concentration in a total volume of 500 μl. The reaction was incubated during 120 min at 30° C. in the dark and stopped by addition of 250 μl of 2 M KOH, after acidifying with 250 μl of 15% (v/v) H2S04. The enzymatic assay has been performed in both aerobic and anaerobic conditions. The acid formed (2-Hydroxyisobutyric acid) was measured by LC-MS.

### 5.5 Activity of Purified Enzymes

Activity of purified enzyme (mUI/mg)

| | |
|---|---|
| Hydroxyisobutyryl-CoA mutase | 2 |
| (with 3-Hydroxybutyryl-CoA 1 mM in | |
| aerobic conditions) | |
| Hydroxyisobutyryl-CoA mutase | 46 |
| (with 3-Hydroxybutyryl-CoA 1 mM in | |
| anaerobic conditions) | |
| Hydroxyisobutyryl-CoA mutase | 223 |
| (with 3-Hydroxybutyryl-CoA 10 mM in | |
| anaerobic conditions) | |

This activity measured in vitro demonstrates that the multimeric active protein obtained from a mixture of IcmAmp, IcmBmp and Coenzyme B12 catalyzes the conversion of 3-hydroxybutyryl-CoA into 2-hydroxyisobutyryl-CoA (converted into 2-hydroxyisobutyric acid in our enzymatic assay by acid hydrolysis). We also demonstrate that hydroxyisobutyryl-CoA mutase is more than 20 times more active under anaerobic conditions.

### 6 Example 6

### Purification of tesB Gene Product of E. coli Encoding for Acyl-CoA Thioesterase, and ptbca and bukca Gene Products of C. acetobutylicum Encoding for Phosphotransbutyrylase and Butyrate-Kinase Activities, and Demonstration that the Gene Products Catalyze the Conversion of 2-Hydroxyisobutyryl-CoA into 2-Hydroxyisobutyric Acid

In order to demonstrate that the acyl-CoA thioesterase activity, or the combination of the phosphotransbutyrylase and butyrate-kinase activity, catalyze the conversion of 2-hydroxyisobutyryl-CoA into 2-hydroxyisobutyric acid, we have purified separately the three tesB, ptbca and bukca genes product, and realized enzymatic assay.

6.1 Construction of the Overexpression Plasmids for tesB, ptbca and bukca
6.1.1 Construction of the Plasmid pPAL7-tesB
To amplify the gene tesB from Escherichia coli, a PCR is carried out using E. coli Genomic DNA as template and the primers named pPAL7-tesB F and pPAL7-tesB R.

pPAL7-tesB F
(SEQ ID NO 33)
CCCAAGCTTTGATGAGTCAGGCGCTAAAAAATTTACTGAC
pPAL7-tesB R
(SEQ ID NO 34)
GGAATTCTTAATTGTGATTACGCATCACCCCTTCC

PCR product is digested with HindIII and EcoRI and cloned into the vector pPAL7 (Profinity eXact pPAL7 Vector Biorad) cut by the same restriction enzymes. The resulting plasmid is named pPAL7-tesB.

Vector pPAL7-tesB is introduced into E. coli BL21 star (DE3) chemically competent cells, leading to strain BL21 star (DE3) (pPAL7-tesB).

6.1.2 Construction of the Plasmid pPAL7VB01-ptbca

To amplify the gene ptb from Clostridium acetobutylicum, a PCR is carried out using C. acetobutylicum Genomic DNA as template and the primers named pPAL7-ptbca F and pPAL7-ptbca R.

pPAL7-ptbca F
(SEQ ID NO 35)
CCCAAGCTTTGACTTCTATGATTAAGAGTTTTAATG
pPAL7-ptbca R
(SEQ ID NO 36)
GGAATTCTTATTTATTGCCTGCAACTAAAGCTGC

PCR product is digested with HindIII and EcoRI and cloned into the vector pPAL7 (Profinity eXact pPAL7 Vector Biorad) cut by the same restriction enzymes. The resulting plasmid is named pPAL7VB01-ptbca. The protein overexpressed brings two adding amino acid (Thr-Ser) which correspond to the spacer between the tag used and the protein in order to ensure successful purification.

Vector pPAL7VB01-ptbca is introduced into E. coli BL21 star (DE3) chemically competent cells, leading to strain BL21 star (DE3) (pPAL7VB0′-ptbca).

6.1.3 Construction of the Plasmid pPAL7VB01-bukca

To amplify the gene buk from Clostridium acetobutylicum, a PCR is carried out using C. acetobutylicum Genomic DNA as template and the primers named pPAL7-bukca F and pPAL7-bukca R.

pPAL-bukca F
(SEQ ID NO 37)
CCCGCTCTTCAAAGCTTTGACTTCTATGTATAGATTACTAATAATCAAT
CC
pPAL-bukca R
(SEQ ID NO 38)
GGAATTCTTATTTGTATTCCTTAGCTTTTTCTTCTCC

PCR product is digested with SapI and EcoRI and cloned into the vector pPAL7 (Profinity eXact pPAL7 Vector Biorad) cut by the same restriction enzymes. The resulting plasmid is named pPAL7VB01-bukca. The protein overexpressed brings two adding amino acid (Thr-Ser) which correspond to the spacer between the tag used and the protein in order to ensure successful purification.

Vector pPAL7VB01-bukca is introduced into E. coli BL21 star (DE3) chemically competent cells, leading to strain BL21 star (DE3) (pPAL7VB01-bukca).

### 6.2 Overproduction of the Acyl-CoA Thioesterase, Phosphatase and Kinase, TesB, Ptbca and Bukca

The overproduction of the proteins TesB, Ptbca and Bukca was done applying the same protocol as example #5.2.

### 6.3 Purification of the Proteins TesB, Ptbca and Bukca

### 6.3.1 Step 1: Preparation of Cell-Free Extracts.

About 100 mg (120 mg for TesB) of E. coli biomass was resuspended in 15 ml of 100 mM potassium phosphate pH 7.6, and a protease inhibitor cocktail. The cell suspension was sonicated on ice (Bandelin sonoplus, 70 W) in a 50 ml conical tube during 8 cycles of 30 sec with 30 sec intervals. After sonication, cells were incubated for 30 min at room temperature with 5 mM MgCl2 and 1 UI/ml of DNaseI. Cells debris were removed by centrifugation at 12000 g for 30 min at 4° C.

### 6.3.2 Step 2: Affinity Purification

Purification of the proteins TesB, Ptbca and Bukca was done applying the same protocol as example 5.3.2 above.

### 6.4 Enzymatic Assay

### 6.4.1 Acyl-CoA Thioesterase Assay

The acyl-CoA thioesterase assay was carried out with 100 mM potassium phosphate buffer pH 8, 0.1 mM 5,5′-dithiobis-2-nitrobenzoic acid (DTNB), 200 μM 2-Hydroxyisobutyryl-CoA and about 11 μg of purified protein in a total volume of 1 ml. The rate of increase in absorbance at 405 nm, was measured at 30° C. on a spectrophotometer. Control assay, lacking the enzyme, was subtracted to the assay. A unit of acyl-CoA thioesterase activity is the amount of enzyme required to hydrolyse 1 μmol of substrate per min. (Epsilon 405 nm=13600 M-1 cm-1)

### 6.4.2 Acid-Kinase Assay

The reaction is carried out in the presence of excess hydroxylamine with 2-Hydroxyisobutyric acid (or 3-Hydroxybutyric acid) and ATP as reactants. The assay method makes use of the ability of acyl phophates to form hydroxamic acids immediately at neutrality and the subsequent formation of the colored ferric-hydroxamate complex in acid solution. The enzyme was assayed in a 100 mM Tris-HCl pH 7.4 buffer, 400 mM Hydroxylamine pH 7.4 neutralized with KOH, 50 mM 2-Hydroxyisobutyric acid, 10 mM ATP pH 7.4 neutralized with KOH, 20 mM MgCl2, 150 mM KCL, 120 mM KOH and about 5 μg of purified enzyme in a total volume of 1 ml. Reaction was started with purified enzyme at 37° C., the reaction was stopped after 30 min by addition of 500 μl of a solution containing 370 mM FeCl3, 20% Trichloroacetic acid and 720 mM HCl. Tube was centrifugated at 10000 g for 15 min at 4° C., the supernatant was stored on ice and the absorbance was measured at 540 nm on a spectrophotometer. Control assay, lacking the enzyme, was subtracted to the assay. (Epsilon 540 nm=169 M-1 cm-1).

### 6.4.3 Phosphotransacylase Assay

The phosphotransacylase assay was carried out with 100 mM potassium phosphate buffer pH 7.4, 0.08 mM 5,5′-dithiobis-2-nitrobenzoic acid (DTNB), 200 μM 2-Hydroxyisobutyryl-CoA and about 2 μg of purified protein in a total volume of 1 ml. The rate of increase in absorbance at 405 nm, was measured at 30° C. on a spectrophotometer Control assay, lacking the enzyme, was subtracted to the assay. A unit of phosphotransacylase activity is the amount of enzyme required to hydrolyse 1 μmol of substrate per min. (Epsilon 405 nm=13600 M-1 cm-1).

### 6.5 Activities of Purified Enzymes

| | | Activity of purified |
|---|---|---|
| | Enzyme | enzyme (mUI/mg) |
| Acyl-CoA thioesterase | TesB | 3837 |
| (with 2-Hydroxyisobutyryl-CoA 200 μM) | | |
| Phoshotransacylase | Ptbca | 224 |
| (with 2-Hydroxyisobutyryl-CoA 200 μM) | | |
| Acid-Kinase | | |
| (with 2-Hydroxyisobutyric acid 50 mM) | Bukca | 2414 |

These activities measured in vitro on purified enzymes demonstrate that both the acyl-CoA thioesterase activity and the combination of the phosphotransacylase and the acid-kinase activity catalyze the conversion of 2-hydroxyisobutyryl-CoA into 2-hydroxyisobutyric acid.

6.6 Activities on Crude Extract of Reference Strain E. coli MG1655

| | Activity of MG1655 |
|---|---|
| | crude extract (mUI/mg) |
| Acyl-CoA thioesterase | 15 |
| (with 2-Hydroxyisobutyryl-CoA 200 μM | |

We demonstrate that the acyl-CoA thioesterase specific activity measured in our reference strain MG1655, due to the chromosomic version of tesB, is sufficient to convert 2-Hydroxyisobutyryl-CoA into 2-Hydroxyisobutyric acid.

### 7 Example 7

### Fermentation of 2-Hydroxyisobutyric Acid Producing Strains in Erlenmeyer Flasks

### 7.1 Fermentation of 2-HIBA Producing Strains in Baffled Erlenmeyer Flasks (Aerobic Conditions)

Performances of strains were initially assessed in 500 ml baffled Erlenmeyer flask cultures using modified M9 medium (Anderson, 1946, Proc. Natl. Acad. Sci. USA 32:120-128) that was supplemented with 10 g/l MOPS, 10 g/l glucose, 10 mg/l B12 vitamin and 100 μM IPTG and adjusted at pH 6.8. Spectinomycin and carbenicillin were added if necessary at a concentration of 50 mg/l and 100 mg/l respectively. A 24 hours preculture was used to inoculate a 50 ml culture to an OD_{600 nm} of about 0.3 to 0.6. (Reducing preculture time enhanced 2-HIBA production of strain HI0048c01). The cultures were kept on a shaker at 37° C. and 200 rpm until the glucose in the culture medium was exhausted. Glucose consumption was followed by HPLC using a Biorad HPX 97H column for the separation and a refractometer for the detection. 2-HIBA production was followed by LC-MS/MS (liquid chromatography-mass spectrometry coupling).

Comparison of the performances of different strains is given in table below.

| | | | | [2-H1BA] |
|---|---|---|---|---|
| Culture_ref | Strain_ref | Deletion(s) | Plasmid(s) | (μg/l |
| FbHI_0077 & | HI0008 | / | (pME101-phaAre-phaBre-TT02) | nd |
| FbHI_0126 | | | | |
| FbHI_0069 & | HI0012 | / | (pME101-icmAmp-icmBmp-TT07-Plac-phaAre- | 85 ± 35 |
| FbHI_091 | | | phaBre-TT02) | |
| FbHI_0092 & | HI0048 | / | (pME101-icmAmp-icmBmp-TT07-Plac-phaAre- | 220 ± 10 |
| FbHI_0114 | | | phaBre-TT02) | |
| | | | (pJB137-fldA-TT07-fpr) | |
| FbHI_0128 | HI0048 | / | (pME101-icmAmp-icmBmp-TT07-Plac-phaAre- | 440 |
| (7h | | | phaBre-TT02) | |
| preculture) | | | (pJB137-fldA-TT07-fpr) | |
| FbHI_0093 & | HI0060 | / | (pUC18-Ptrc01-icmAmp-icmBmp-TT07-Plac- | 150 ± 10 |
| FbHI_0115 | | | phaAre-phaBre-TT02) | |
| FbHI_0095 & | HI0062 | ΔudhA | (pUC18-Ptrc01-icmAmp-icmBmp-TT07-Plac- | 185 ±15 |
| FbHI_0131 | | Δpgi | phaAre-phaBre-TT02) | |

nd : not detected

### 7.2 Fermentation of 2-HIBA Producing Strains in Non-Baffled Erlenmeyer Flasks (Micro-Aerobic Conditions)

As IcmAmp/IcmBmp enzymatic complex has been shown to be more efficient in absence of oxygen (results obtained with in vitro enzymatic assay see example #5.5), initial protocol has been modified to apply a micro-aerobic step of culture. Performances of strains were then assessed in 500 ml non-baffled Erlenmeyer flasks using modified M9 medium, that was supplemented with 20 g/l MOPS, 10 g/l glucose and 100 μM IPTG and adjusted at pH 6.8. Spectinomycin and carbenicillin was added if necessary at a concentration of 50 mg/l and 100 mg/l respectively. A 24 hours preculture was used to inoculate a 50 ml culture to an OD_{600 nm} of about 0.3 to 0.6. The cultures were kept on a shaker at 37° C. and 200 rpm until OD_{600 nm} was higher than 4. Then 10 mg/l B12 vitamin was added in the culture medium and shaker agitation speed was decreased to 100 rpm until the glucose in the culture medium was exhausted. Glucose consumption was followed by HPLC using a Biorad HPX 97H column for the separation and a refractometer for the detection. 2-HIBA production was followed by LC-MS/MS (liquid chromatography-mass spectrometry coupling).

Comparison of the performances of different strains is given in table below.

| Culture_ref | Strain_ref | Deletion(s) | Plasmid(s) | [2-H1BA] (μg/l) |
|---|---|---|---|---|
| FbHI_0127 | HI0008 | / | (pME101-phaAre-phaBre-TT02) | nd |
| FbHI_0096 | HI0012 | / | (pME101-icmAmp-icmBmp-TT07-Plac- | 120 |
| | | | phaAre-phaBre-TT02) | |
| FbHI_0097 | HI0048 | / | (pME101-icmAmp-icmBmp-TT07-Plac- | 240 |
| | | | phaAre-phaBre-TT02) | |
| | | | (pJB137-fldA-TT07-fpr) | |

nd : not detected.

### 7.3 Impact of B12 Vitamin on 2-HIBA Production of Strain HI0048 in Baffled Erlenmeyer Flasks (Aerobic Conditions)

As IcmAmp/IcmBmp enzymatic complex is known to be B12-vitamin dependant, two cultures of strain HI0048c01 have been performed with or without B12-vitamin supply, in culture conditions described in example #7.1, with a 7 hours preculture instead of 24 hours one. Glucose consumption was followed by HPLC using a Biorad HPX 97H column for the separation and a refractometer for the detection. 2-HIBA production was followed by LC-MS/MS (liquid chromatography-mass spectrometry coupling). 2-HIBA production is given in FIG. 1.

Performances are given in table below.

| | | [B12- | | [2- |
|---|---|---|---|---|
| | | vitamin] | | HIBA] |
| Culture_ref | Strain ref | (mg/L) | Plasmid(s) | (μg/l) |
| FbHI_0128 | HI0048 | 10 | (pME101- | 440 |
| | | | icmAmp-icmBmp- | |
| | | | TT07-Plac- | |
| | | | phaAre-phaBre-TT02) | |
| | | | (pJB137-fldA-TT07-fpr) | |
| FbHI_0129 | HI0048 | 0 | (pME101- | nd |
| | | | icmAmp-icmBmp-TT07-Plac- | |
| | | | phaAre-phaBre-TT02) | |
| | | | (pJB137-fldA-TT07-fpr) | |

nd : not detected

1. A method for the preparation of 2-hydroxyisobutyric acid (2-HIBA) comprising the successive steps allowing conversion of acetyl-CoA into 2-hydroxyisobutyric acid, said successive steps consisting in:
a) converting acetyl-CoA into 3-hydroxybutyryl-CoA
b) converting 3-hydroxybutyryl-CoA previously obtained into 2-hydroxyisobutyryl-CoA, and
c) converting 2-hydroxyisobutyryl-CoA into 2-hydroxyisobutyric acid.
wherein the steps a), b) and c) are enzymatic conversions.

2. The method of claim 1, wherein the enzymatic activity in step a) is obtained with the combination of two enzymes,
the first enzyme a1) having an acetoacetyl-CoA thiolase or acetyl-CoA acetyl-transferase activity and
the second enzyme a2) having a 3-hydroxybutyryl-CoA dehydrogenase activity.

3. The method of claim 2, wherein enzyme a1) is a gene product encoded by genes selected among the group consisting of atoB of E. coli, thlA of C. acetobutylicum and phaA of R. eutropha.

4. The method of claim 2, wherein enzyme a2) is a gene product encoded by genes selected from the group consisting of hbd of C. acetobutylicum and phaB of R. eutropha.

5. The method of claim 1, wherein step b) is obtained with an enzymatic system having a hydroxyisobutyryl-CoA mutase activity.

6. The method of claim 5, wherein the hydroxyisobutyryl-CoA mutase activity is performed by enzymes resulting from the gene products encoded by the genes icmA and icmB from A. tertiaricarbonis, from M. petroleiphilum or from Streptomyces spp.

7. The method of claim 6 wherein the activity of the hydroxyisobutyryl-CoA mutase is increased by overexpressing the fldA-fpr activation system.

8. The method of claim 1, wherein step c) is obtained by transfer of CoA on a substrate with an enzyme having a CoA transferase activity.

9. The method of claim 8, wherein the enzyme has an acetyl-CoA transferase activity and the substrates are acetate and 2-hydroxyisobutyryl-CoA.

10. The method of claim 1, wherein step c) is obtained by transfer of CoA on a substrate with an enzyme having an acyl-CoA thioesterase activity.

11. The method of claim 10, wherein the acyl-CoA thioesterase activity is performed by enzymes resulting from the gene products encoded by genes selected from the group consisting of tesB of E. coli and ybgC from H. influenzae.

12. The method of claim 1, wherein step c) is obtained with the combination of two enzymes
the first enzyme c1) having a phosphotransacylase activity and
the second enzyme c2) having an acid-kinase activity.

13. The method of claim 12, wherein enzyme c1) has a phosphate hydroxyisobutyryltransferase activity, which is optionally a gene product encoded by gene ptb of C. acetobutylicum.

14. The method of claim 12, wherein enzyme c2) is a hydroxyisobutyrate kinase, which is optionally a gene product encoded by gene buk of C. acetobutylicum.

15. The method of claim 1, wherein acetyl-CoA is obtained by bioconversion of a source of carbon in a microorganism.

16. The method of claim 1, wherein steps a), b) and c) are performed by a microorganism expressing the genes coding for the enzymes having the enzymatic activities necessary for the conversions of said steps a), b) and c).

17. The method of claim 1, wherein steps a), b) and c) are performed by the same microorganism.

18. The method of claim 17, wherein the same microorganism provides for the bioconversion of glucose into acetyl-CoA.

19. A microorganism modified for an improved production of 2-hydroxyisobutyric acid, wherein said microorganism expresses the genes coding for the enzymes having the enzymatic activities necessary for the following conversions
a) converting acetyl-CoA into 3-hydroxybutyryl-CoA
b) converting 3-hydroxybutyryl-CoA previously obtained into 2-hydroxyisobutyryl-CoA, and
c) converting 2-hydroxyisobutyryl-CoA into 2-hydroxyisobutyric acid.

20. The microorganism of claim 19 which is modified to produce higher levels of acetyl-CoA.

21. The microorganism of claim 20, wherein the expression of at least one of the following genes is attenuated:
pta encoding phospho-transacetylase
ackA encoding acetate kinase
poxB encoding pyruvate oxidase
ldhA encoding lactate dehydrogenase
aceA encoding isocitrate lyase.

22. The microorganism of claim 19, wherein the availability of NADPH is increased.

23. The microorganism of claim 22, wherein the expression of at least one of the following genes is attenuated:
pgi encoding the glucose-6-phosphate isomerase
udhA encoding the soluble transhydrogenase.

24. The microorganism of claim 19 comprising a bacteria, optionally selected from the group consisting of Enterobacteriaceae, Clostridiaceae, Bacillaceae, Streptomycetaceae and Corynebacteriaceae.

25. A method for the fermentative production of 2-hydroxyisobutyric acid (2-HIBA) by conversion of a simple source of carbon into 2-HIBA comprising the steps of:
culturing the microorganism of claim 19 in an appropriate culture medium comprising a simple source of carbon, and
recovering the 2-hydroxyisobutyric acid (2-HIBA) from the culture medium.

26. The method of claim 25, wherein the 2-hydroxyisobutyric acid (2-HIBA) is further purified.
